(19) United States
(12) Patent Application Publication
(10) Pub. No.: US 2016/0128731 A1
(43) Pub. Date: May 12, 2016

(54) OSTEOSYNTHESIS DEVICE WITH CERVICO-CEPHALIC SCREW

(71) Applicants: Laurent FUMEX, Madison, CT (US); Thierry MASSEGLIA, La Garde (FR)

(72) Inventors: Laurent FUMEX, Madison, CT (US); Thierry MASSEGLIA, La Garde (FR)

(21) Appl. No.: 14/894,747

(22) PCT Filed: May 22, 2014
(86) PCT No.: PCT/FR2014/051206

§ 371 Date: Nov. 30, 2015

(30) Foreign Application Priority Data
May 28, 2013 (FR) 1354841

(51) Int. Cl. A61B 17/68; A61B 17/80

(52) U.S. Cl. CPC A61B 17/683; A61B 17/8061

(57) ABSTRACT

The invention relates to an osteosynthesis device.

The osteosynthesis device comprises a cervico-cephalic screw (1) provided with a distal thread able to be screwed into the bone, a support (3) formed by a plate with a shape suitable for fixing same to the bone or by an intramedullay nail able to be inserted into the medullary cavity, and a blocking means (6) blocking the screw in the support and movable in the support along the axis of the screw, and the final positioning of the screw is variable.

The device is intended for holding in place and compressing fragments of a fractured bone.

The present invention relates to a device for holding in place and compressing fragments of a fractured bone.

Many devices are known which are intended to maintain compression of fragments of a fractured bone, for example of a femoral neck. The patent U.S. Pat. No. 4,612,920 describes a plate having, at its distal end, an oblong bore which makes it possible to block the rotation of a cervico-cephalic screw having, at its distal end, a thread of the cancellous bone type and, at its proximal end, a cylindrical body comprising two flat parts. The compression of the fracture site and the blocking in one direction are effected by means of a compression screw which is screwed into the cervico-cephalic screw and bears in a bore made in the proximal end of the barrel of the plate. The patent U.S. Pat. No. 5,484,439 describes a screw/plate using the same type of screws housed in the barrel of the plate. Devices of this “screw/plate” type are still used today.

The application WO 2008/128663 A2 describes a device for reduction and compression adapted to the fracture of the femoral neck, also having a plate screwed into the bone of the femur and carrying two or three compression screws parallel to the axis of the femoral neck. A ring or a nut is placed on the head of the compression screw in order to block it in the hole of the plate and to prevent possible axial movement under the weight of the body. An example of an intramedullary nail associated with a compression screw for reduction of a fracture of the head of a femur is described in the patent EP 1 443 865 B1.

The application US 2013/0041414 A1 discloses a telescopic screw being screwed into a bone nail which is inserted in the femur. The telescopic screw has a blocking ring that is able to deform in order to fix the position of the screw in the nail. Similar systems are described in the application DE 197 23 339 A1 and the application EP 1 415 605 A1. A bone nail able to receive a compression screw in a desired position is described in WO 2012/099944 A1.

These devices do indeed allow fragments of a fractured bone to be held in place and compressed, but they require a complicated application technique and a degree of know-how that only an experienced practitioner possesses. More particularly, the choice of the correct length of the cervico-cephalic screw is important, since the very principle of these devices rests on the ability of one piece to slide in the other. The literature has described extensively the complications that arise as a result of the screw being too short, or becoming too short, or being too long: detachment of the screw and plate, or blocking in translation as a result of the screw wedging in the barrel of the plate if the screw is not sufficiently engaged in the plate; course of natural impaction under the weight of the body not controlled; immediate abutment, as from the post-operative phase, of the cephalic screw against the plate, preventing any future sliding; protrusion of the head of the cephalic screw or of the compression screw into the soft parts during the course of natural impaction of the cephalic screw by the weight of the body; excessive shortening of the lower limb following too great an impaction of the cephalic screw; loss of anchoring of the thread of the cephalic screw in the bone during excessive manual compression. The difficulty in determining the correct length of screw is therefore real and the source of complications; it is of course made worse in the case of multi-fragment fractures or unstable fractures whose reduction is difficult. The dimensions of the fractured bone may then vary during the placement of the screw/plate, when the choice of screw has already been made. The operator may attempt to correct these variations by impaction of the plate or by manual compression of the cephalic screw, but in any event an inexperienced operator will not know precisely what will finally be the exact course of impaction of the cephalic screw.

Moreover, during the placement of a device of the screw/plate type comprising, as is most often the case, a cephalic screw with 2 flat parts, the practitioner must orient the two flat parts of the body of the cervico-cephalic screw perfectly in relation to the axis of the femur and slide the plate thereof in order to be able to fix it correctly on the femur; this manoeuvre complicates the operating technique and increases the risk of adversely affecting the quality of the resulting reduction of the fracture site and the anchoring of the screw in the bone.

Finally, in order to adapt as best as possible to the surgical constraints, the manufacturer of these devices has to provide the practitioner with numerous cervico-cephalic screws, of which the length may vary from 50 mm to 145 mm in increments of 5 mm, which entails high costs of production and storage, both for the manufacturer and also for the hospital.

The present invention relates to an osteosynthesis device allowing two parts of a fractured bone to be held in place and compressed, simplifying the operating technique, permitting variable final positioning of the screw in the plate, making it possible to neutralize the approximation of measurements arising from the surgical constraints, suppressing the risks of detachment and of wedging, guaranteeing a constant value, predefined by the manufacturer, of the course of impaction of the cephalic screw irrespective of its position in the plate, and significantly reducing the range of cervico-cephalic screws that are needed, without reducing the operating possibilities.

The device according to the present invention, which can be used in particular for fractures of the ends of the femur, is of the type mainly comprising a) a cervico-cephalic screw provided with a distal thread, preferably sliding on the same body of the screw that can be screwed into the bone, b) a support formed by a plate with a shape suitable for fixing same to the bone or by an intramedullary nail able to be inserted into the medullary canal, and c) a blocking means for blocking the screw on the support, and it is characterized by the fact that the blocking means for blocking the cervico-cephalic screw is movable on the support along the axis of the screw, and in that the final positioning of the screw is variable.

The blocking means is preferably formed by a clamping screw acting on an elastically deformable ring that can be blocked by friction against the wall of the support retaining the cervico-cephalic screw.

According to the invention, the ring has a conical shape, of which the wall has at least one slot, cooperating with the proximal end of the cervico-cephalic screw, and comprises fixing means for fixing on the clamping screw. According to a variant, the ring is formed by an elastically deformable bead formed in the proximal part of the body of the cervico-cephalic screw.

According to a first embodiment, the cervico-cephalic screw comprises two distinct elements connected by fixing means, namely a threaded screw head forming the distal part, and a screw body, of which the proximal part receives the blocking means in the support.

Preferably, the screw head comprises means for sliding on the screw body while blocking rotation, that is to say without possibility of rotation, such that the body and the screw head are integrally connected in rotation but not in translation.

According to one feature of the invention, the screw body has, at its distal part, an elastically deformable means that is able to engage with a snap fit in the screw head. This elastically deformable means is able to deform under a force of defined value that frees the translation of the screw head when a force applied axially to the screw head exceeds this predefined value. Preferably, the translation of the screw head with respect to the screw body is limited by at least two stops.

According to one embodiment, the screw body comprises, at its distal end, a slotted head engaging with a snap fit in the screw head, a cylindrical distal part having two flat parts cooperating with the inner proximal part of the screw head, a central cylindrical part with a diameter greater than the diameter of the distal cylindrical part, sliding in the inner hole of the support plate, a proximal part of conical shape cooperating with the inner conical part of the slotted ring, having an inner thread cooperating with that of the clamping screw and two flat parts cooperating with the application instrument.

According to an advantageous embodiment of the invention, the proximal end of the screw body is composed of a Morse taper with a conicity of preferably between 1% and 20%.

The head of the cervico-cephalic screw is formed, at its outer distal end, by a thread of the cancellous bone type; at its inner distal end, by a throat and a bore cooperating with the slotted overthickness of the screw body; at its outer proximal end, by a cylindrical part of smaller diameter than the hole of the plate; at its inner proximal end, by a cylindrical with two flat parts cooperating with the distal part of the screw body. The length of the inner cylindrical part that is able to slide on the screw body is preferably greater than about 2.5 times the value of its diameter, in order to avoid any risk of it becoming stuck by wedging.

The slotted ring, slotted axially in order to permit plastic and elastic deformation, has, at its inner distal end, a conical bore which cooperates with that of the proximal end of the bone screw and, at its proximal end, a shoulder which engages with a snap-fit action on the clamping screw so as to block the device once it is in place.

The clamping screw is composed, at its distal end, of an outer thread cooperating with that of the distal end of the screw body, and, at its proximal end, of a means of attachment to the ring.

According to one advantageous embodiment, the external diameters of the outer proximal part of the screw head, of the central part of the screw body, of the slotted ring and of the head of the clamping screw are less than the internal diameter of the barrel of the plate holding the cervico-cephalic screw.

According to a preferred embodiment, the shoulder of the distal slotted head of the screw body and the shoulder formed by the difference in diameter between the distal part and the central part of the screw body limit the translation of said screw head to a value of between 10 and 16 mm and preferably about 13 mm.

The constituent elements of the device of the invention, that is to say the cervico-cephalic screw, the conical ring, the clamping screw and the plate, or the intramedullary nail if appropriate, are made of materials currently used in osteosynthesis devices, preferably of stainless steel or of titanium.

The advantages of the present invention will become clearer after the following explanation of its fitting. The practitioner, using the customary techniques, prepares the bone cavity by drilling it and tapping it, then chooses the cervico-cephalic screw that is suitable depending on the measured length. He introduces this cervico-cephalic screw into the plate, then screws this assembly into the bone by means of a customary screwing instrument of the spanner type, using a guide pin on which the cervico-cephalic screw is engaged. Once the screwing has been completed, and after removal of the pin if appropriate, the practitioner orients the plate with respect to the femur, strikes it with an instrument of the impactor type, then fixes it by means of cortical screws. Finally, he screws the assembly, with the slotted ring snap-fitted on the clamping screw, into the screw body. During the screwing procedure, the head of the clamping screw pushes the slotted ring, which deforms by sliding on the Morse taper of the screw body, thereby increasing its external diameter until it is blocked in rotation and translation against the wall of the inner hole of the plate. When the screwing procedure is finished, the screw body is blocked in rotation by the friction of the cones and blocked in translation in the ring, which is itself blocked in the plate.

Under the effect of the natural impaction by the weight of the body, the screw head will be able to slide by a maximum of 13 mm with respect to the plate without protrusion of the blocking screw.

Since the translation of the screw head with respect to the screw body is ensured without risk of wedging, a cervico-cephalic screw can be blocked in a plate in any depth of screwing in the bone, for example from 80 to 110 mm for a length of barrel of the plate of 42 mm. This simplifies the operating procedure, allows for imprecision and reduces the number of cervico-cephalic screws to two for a range covering the lengths of 65 to 110 instead of the ten cervico-cephalic screws normally proposed with the customary devices.

The device is easy to remove since, when the clamping screw is unscrewed, it entrains and unseats the slotted ring from inside the plate, permitting disassembly of the cervico-cephalic screw by means of a T-shaped key.

Other features and advantages of the present invention will become clear from the following description which relates to a preferred embodiment and in which reference is made to the attached figures, in which:

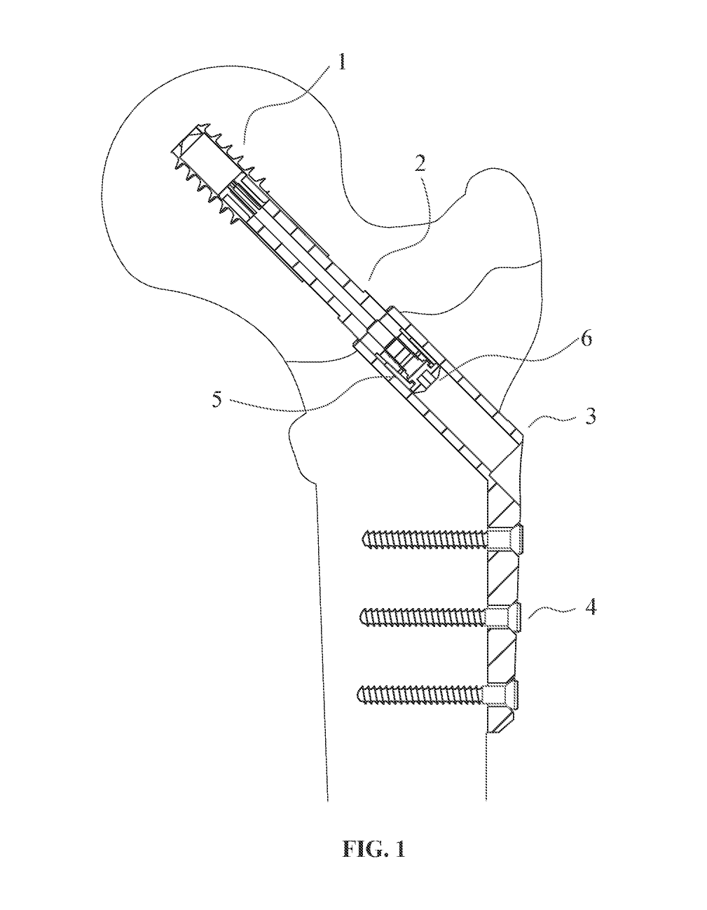

FIG. 1 shows a sectional view of the device mounted in a plate, the whole assembly being implanted in a femur.

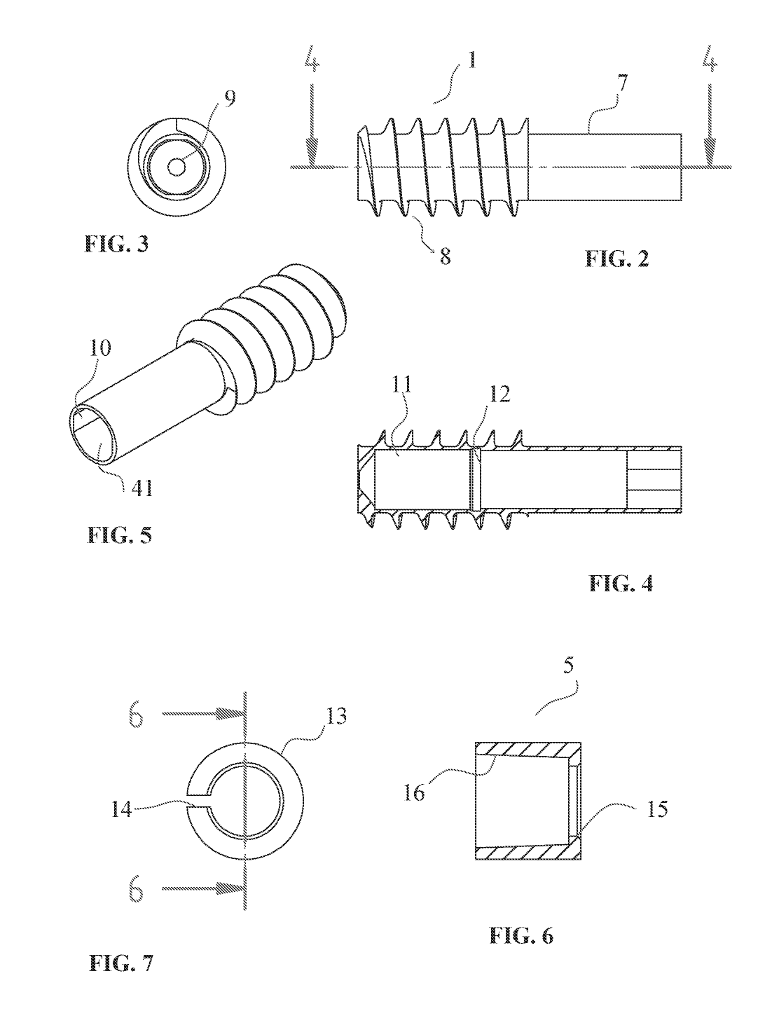

FIGS. 2 to 5 show views defining the cervico-cephalic screw head.

FIGS. 6 to 7 show views defining the slotted ring.

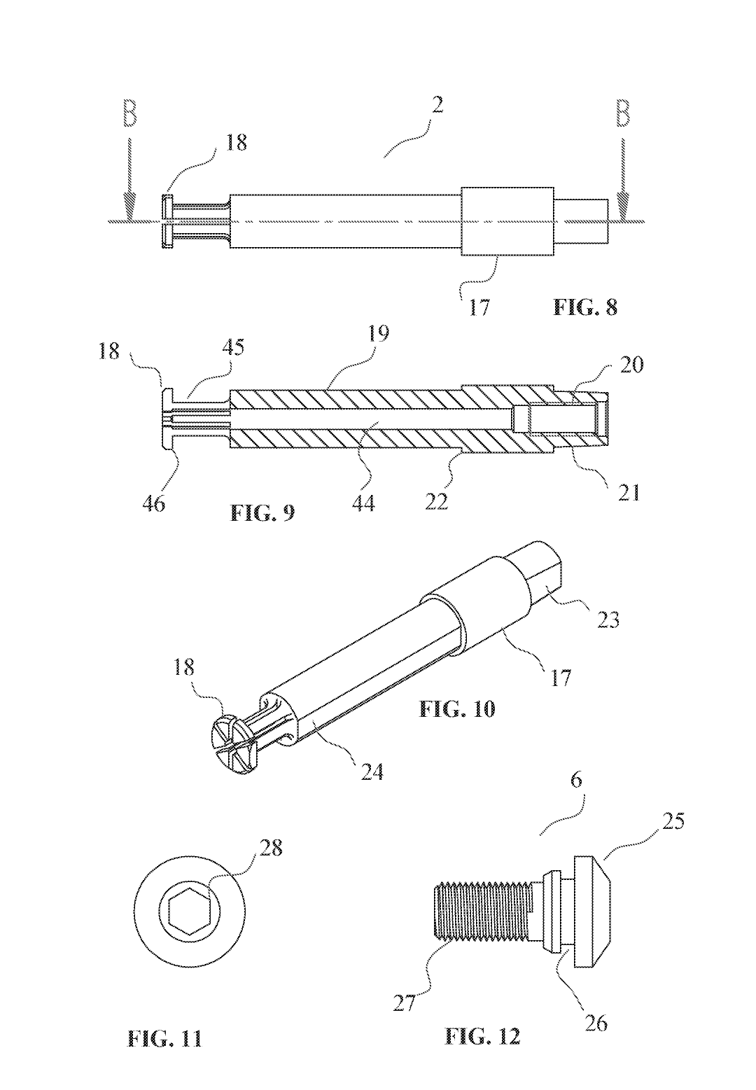

FIGS. 8 to 10 show views defining the cervico-cephalic screw body.

FIGS. 11 to 12 show views defining the clamping screw.

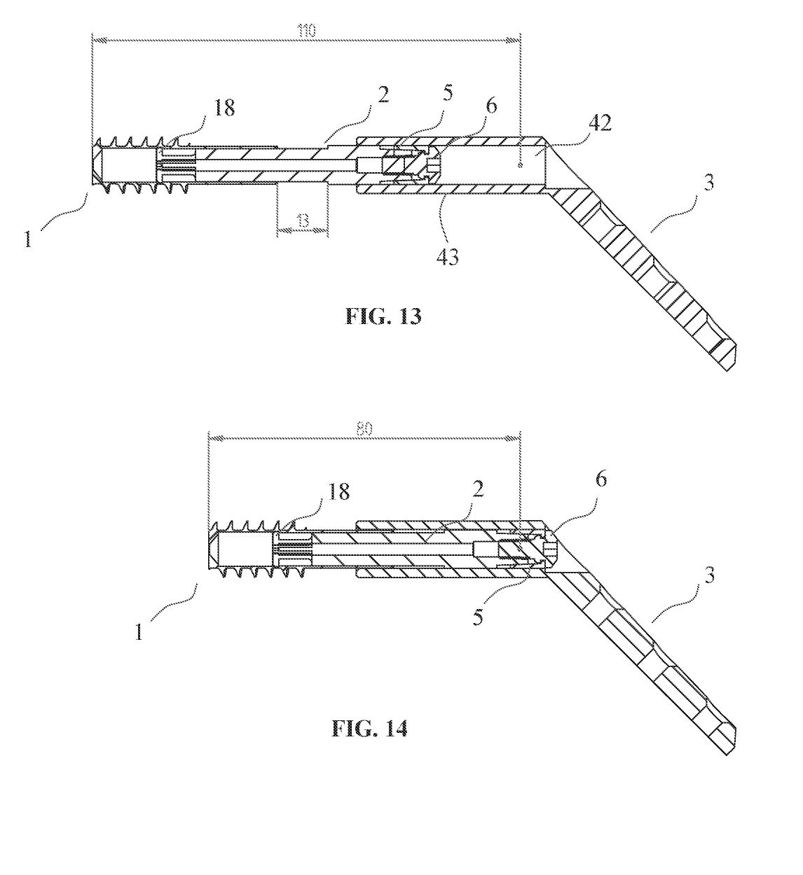

FIGS. 13 to 14 show sectional views of the device mounted in a plate indicating the maximum and minimum positions of the same screw.

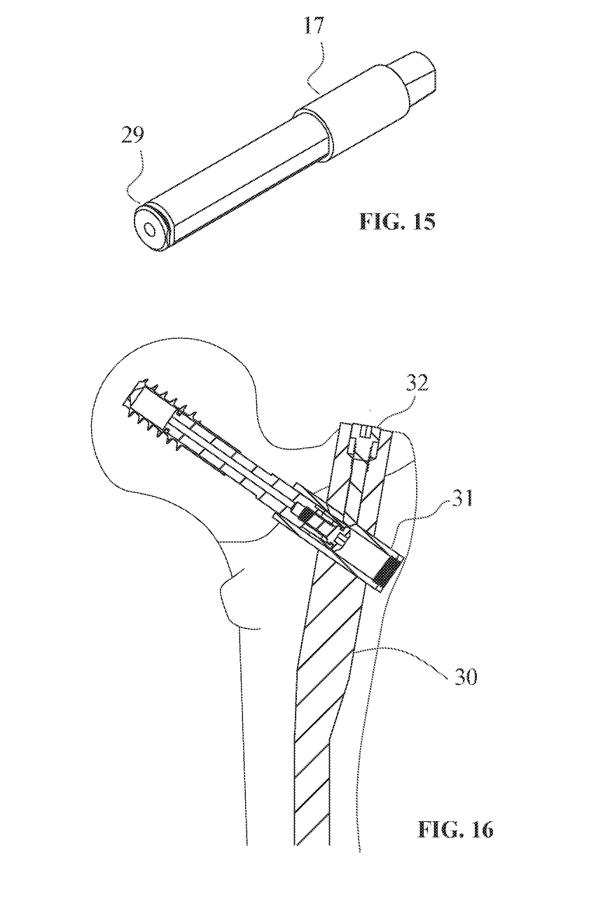

FIG. 15 shows a perspective view of a variant of the cervico-cephalic screw body.

FIG. 16 shows a plan view of the device mounted in a plate, the whole assembly being implanted in an intramedullary nail.

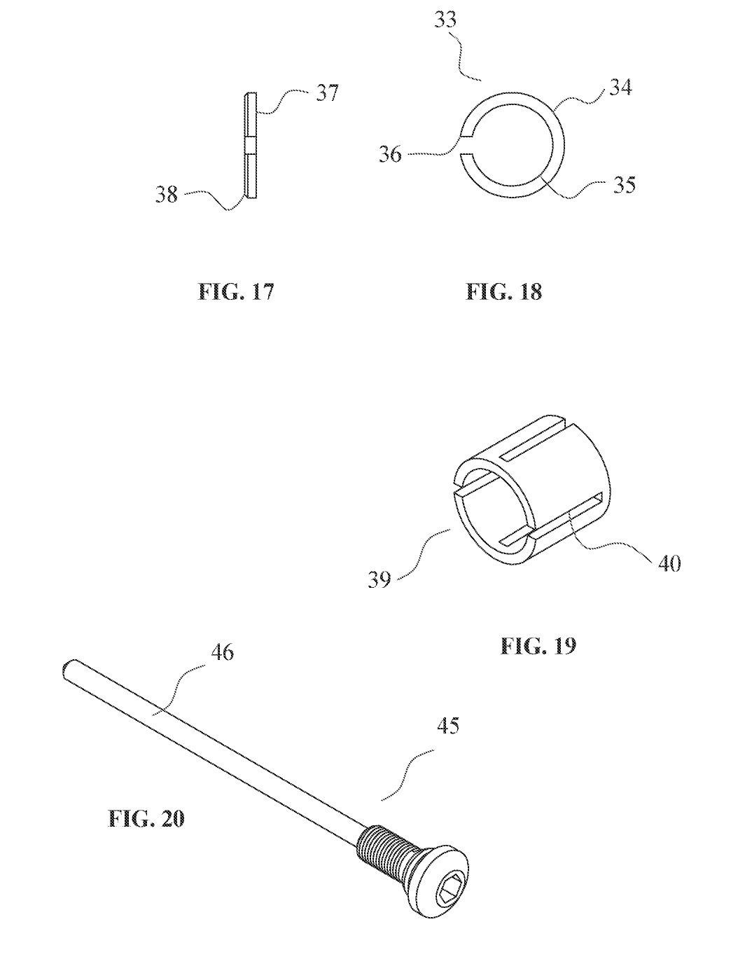

FIGS. 17 to 18 show views defining the slotted ring.

FIG. 19 shows a perspective view of a variant of the slotted ring.

FIG. 20 shows a perspective view of a variant of the damping screw.

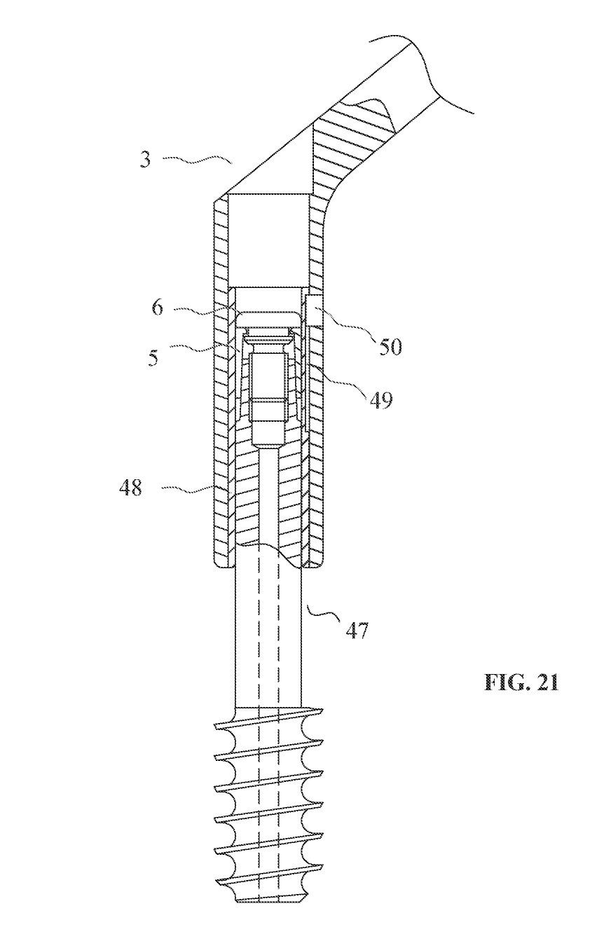

FIG. 21 shows sectional views of a variant of the device mounted in a plate.

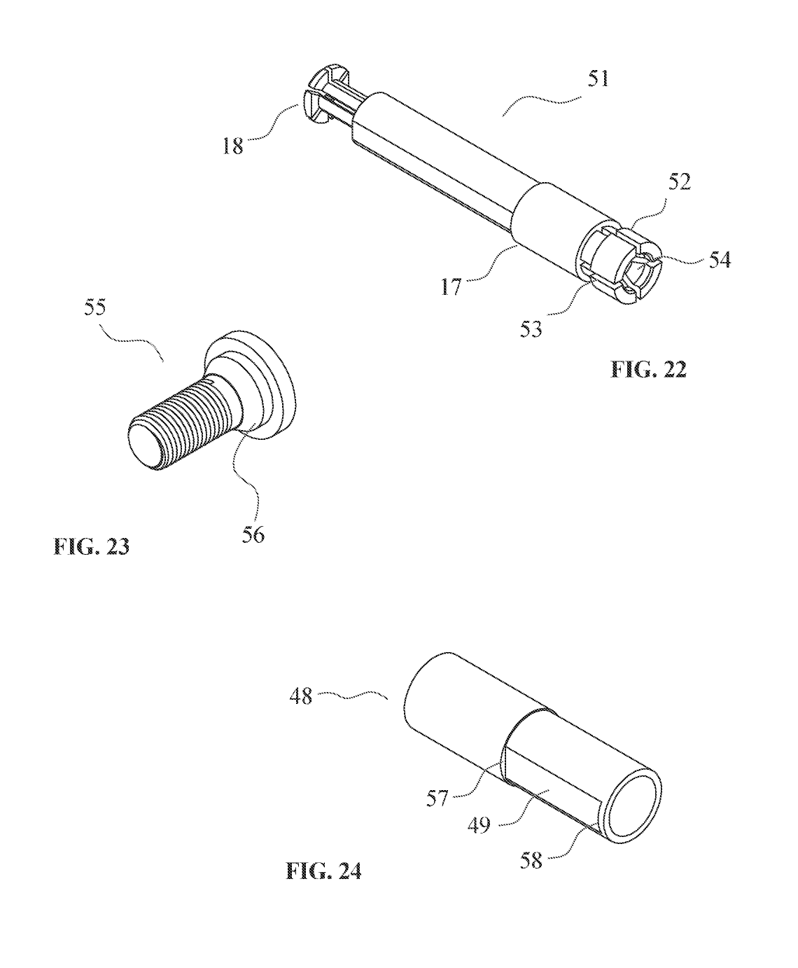

FIG. 22 shows a perspective view of a variant of the cervico-cephalic screw body.

FIG. 23 shows a perspective view of a variant of the damping screw.

FIG. 24 shows a perspective view of a detail from FIG. 21.

The cervico-cephalic screw shown in FIG. 1 is composed of the screw head (1) mounted on the screw body (2), and it is mounted in a plate (3) fixed on the femur by means of cortical screws (4).

The screw head (1), shown in FIGS. 2 to 5, comprises, at its outer distal end, a thread (8) of the cancellous bone type which permits its anchoring in the bone. The inner distal end is composed of a bore (11), in which the slotted head (18) of the screw body slides with friction, and of a throat (12) in which the head (18) of the screw body can engage with a snap fit. The proximal part is composed of a bore (41) with two flat parts (10) cooperating with the cylindrical part (19) and the two flat parts (24) of the screw body. Its diameter (7) is smaller than the bore (42) of the plate (3). The hole (9) allows it to be guided on a pin during fitting.

The slotted ring (5), shown in FIGS. 6 and 7, has a conical bore (16) cooperating with the cone (21) of the screw body (2). It has a groove (14) slitting it axially, and a shoulder (15) engaging in the throat (26) of the clamping screw (6). Its external diameter (13) is smaller than the bore (42) of the plate (3).

The screw body (2), shown in FIGS. 8 to 10, has, at its distal end, a slotted head (18). During the assembly of the cervico-cephalic screw, and by virtue of its elastic deformation, the slotted head (18) of the screw body, after closing in order to enter the proximal part (10+41), deploys in the throat (12) of the screw head (1), holding it in this position during the fitting by the practitioner. When the natural impaction generated by the weight of the body applies to the screw head, the slotted head (18) will close and allow the screw head (1) to slide. The force starting from which the screw head (1) will be able to slide is determined according to the difference between the diameters of the slotted head (18) and of the bore (11) and also according to the thickness (45) of the flexible part of the slotted head (18). The screw body (2) has a cylindrical part (19) and two flat parts (24) which cooperate with the diameter (41) and the two flat parts (10) of the screw head (1) in such a way as to permit the sliding movement while blocking rotation. The cylindrical guide (17) slides in the bore (42) of the plate (3). The proximal end has a cone (21) on which two flat parts (23) are formed symmetrically opposite, permitting driving in rotation during the screwing into the bone by means of an instrument. The inner thread (20) ensures the screwing of the damping screw (6), The hole (44) permits its guiding on a pin during the fitting. The shoulders (46) and (22) limit the sliding of the screw head (1) to 13 mm.

The clamping screw (6), shown in FIGS. 11 and 12, has a thread (27) cooperating with the thread (20) of the screw body (2). The throat (26) permits the snap-fit engagement of the ring (5). The head (25) has a diameter smaller than the bore (42) of the plate (3), and a drive means (28) of the hexagon socket type.

FIGS. 13 and 14 show the end positions of the same cervico-cephalic screw mounted in the barrel (43) of a plate (3) while guaranteeing the natural impaction course of 13 mm without protrusion of the clamping screw (6).

The perspective view in FIG. 15 shows a variant of the screw body in which the slotted head has been replaced by a throat (29) configured to receive the slotted ring (33), where the principle of freeing the sliding movement of the screw head (1) at a defined value remains unchanged.

The sectional view in FIG. 16 shows the device mounted in a tube (31), which is blocked by a screw (32) screwed inside an intramedullary nail (30) of a commercially available type. The functions of the device remain unchanged, and the dimensions can be adapted to this configuration. Of course, several devices could be inserted into the intramedullary nail depending on the dimensions.

The slotted ring (33), shown in FIGS. 17 and 18, has a slot (36) and a hole (35), of which the diameter is greater than the diameter of the throat (29), in such a way that it is able to close. Its diameter (34) cooperates with the diameter of the throat (12). The thickness (37) is less than the width of the throat (29), and the chamfer (38) facilitates its deformation during the sliding of the screw head (1).

The perspective view in FIG. 19 shows a variant (the slotted ring (5), having at least two partial grooves (40) distributed in a staggered arrangement on the periphery and facilitating its deformation.

The perspective view in FIG. 20 shows a variant (45) of the clamping screw (6), having a cylindrical part (46) which fits in the hole (44) of the screw body (2) in such a way as to block the deformation of the head (18). This configuration is useful for avoiding the natural impaction by the weight of the body in young patients.

The sectional view in FIG. 21 shows a variant (47) of the cervico-cephalic screw which is in one piece. This cervico-cephalic screw is blocked by means of the slotted ring (5) snap-fitted on the clamping screw (6) in a tube (48) mounted in the plate (3). This tube (48) slides in translation in the plate (3) and is blocked in rotation by way of the stud (50) integrally connected to the plate (3) cooperating with the flat part (49) of the tube (48). The sliding distance is limited by the length of the flat part (49), In FIG. 21, the tube (48) holding the cervico-cephalic screw (47) is in the end position protruding to the greatest extent from the plate (3). This solution makes it possible to reduce the number of cervico-cephalic screws required for adapting to all situations, compared to a known device, even if the range of proposed screws is greater than in the preferred solution with a two-part screw.

The perspective view in FIG. 22 shows a variant (51) of the screw body in which the proximal part (52) is slotted with grooves (53) making it deformable. The conical part (54) cooperates with the cone (56) of the damping screw (55). While the clamping screw (55) is being screwed into the screw body (51), the proximal part (52) sees its external diameter increase under the effect of the penetration of the cone (56), until it is blocked in the inner wall (42) of the plate (3). The screw body is then blocked in rotation and in translation.

The perspective view in FIG. 23 shows a variant (51) of the clamping screw having a conical part (56) which cooperates with the conical part (54) of the screw body.

The perspective view in FIG. 24 shows the tube (48) of the device from FIG. 21, comprising a flat part (49) in sliding contact with the stud (50), preventing any rotation in the support. At each of the two ends of the flat part (49), a stop (57/58) is provided in order to limit the sliding of the tube (48), the stud (50) being able to slide against the flat part (49) between the two stops.

1. Osteosynthesis device for holding in place and compressing fragments of a fractured bone, comprising:
a cervico-cephalic screw provided with a distal thread able to be screwed. into the bone;
a support formed by a plate with a shape suitable for fixing same to the bone or by an intramedullary nail able to be inserted into the medullary cavity; and
a blocking means blocking the screw in the support,
wherein the Hocking means for blocking the cervico-cephalic screw is movable in the support along the axis of the screw, and in that the final positioning of the screw is variable.

2. Device according to claim 1, wherein the blocking means is formed by a clamping screw acting on an elastically deformable ring that can be blocked by friction against the wall of the support holding the cervico-cephalic screw in place.

3. Device according to claim 2, wherein the ring has a conical shape, of which the wall has at least one slot, cooperating with the proximal end of the cervico-cephalic screw, and comprises fixing means for fixing on the clamping screw.

4. Device according to claim 2, wherein the ring is formed by an elastically deformable bead formed in the proximal part of the cervico-cephalic screw body.

5. Device according to claim 1, wherein the cervico-cephalic screw comprises two distinct elements connected by fixing means, namely a threaded screw head forming the distal part, and a screw body, of which the proximal part receives the blocking means on the support.

6. Device according to claim 5, wherein the screw head comprises means for sliding on the screw body while blocking rotation.

7. Device according to claims 5, wherein the screw body has, at its distal part, an elastically deformable means which is able to engage with a snap fit in the screw head.

8. Device according to claim 7, wherein the elastically deformable means is a slotted head which closes under the natural impaction generated by the weight of the body on the screw head, allowing the latter to slide, starting from a defined force value freeing the translation of the screw head.

9. Device according to claim 5, wherein the translation of the screw head is limited by at least two stops and.
